(19) United States
(12) Patent Application Publication
(10) Pub. No.: US 2019/0255295 A1
(43) Pub. Date: Aug. 22, 2019

(54) GUIDEWIRE WITH CONTROLLABLE STIFFNESS

(71) Applicant: Cook Medical Technologies LLC, Bloomington, IN (US)

(72) Inventor: Ralf Spindler, Solsberry, IN (US)

(73) Assignee: Cook Medical Technologies LLC, Bloomington, IN (US)

(21) Appl. No.: 16/281,376

(22) Filed: Feb. 21, 2019

Related U.S. Application Data
(60) Provisional application No. 62/633,742, filed on Feb. 22, 2018.

(51) Int. Cl. A61M 25/09

(52) U.S. Cl. CPC A61M 25/09; A61L 2400/16; A61M 2025/0915; A61M 2025/09083

(57) ABSTRACT

The disclosure generally relates to a guidewire with controllable stiffness and to a method of use thereof. A distal section of the guidewire includes a core wire, a shape memory coil, and a distal element electrically connecting the core wire to the shape memory coil. The shape memory coil has an expanded configuration with smaller stiffness and a compressed configuration having larger stiffness. Upon passage of an electric current through the shape memory coil, the shape memory coil may switch between the expanded and compressed configurations, thus changing the stiffness of the distal section of the guidewire.

### RELATED APPLICATION

This application claims priority to Provisional Patent Application No. 62/633,742, filed on Feb. 22, 2018, which is incorporated by reference in its entirety.

### BACKGROUND

The present disclosure relates to guidewires. In particular, the present disclosure relates to intravascular guidewires with controllable stiffness.

### Background Information

The diagnosis and treatment of diseases in vessels with atherosclerotic plaques (peripheral artery disease, coronary artery disease, cerebrovascular disease, aneurysm) commonly involves the intravascular access via catheterization. Percutaneous catheterization of central veins and arteries is a routine technique. Every year, more than 5 million central venous catheters are inserted in the U.S. An example of this in the cardiovascular field is the Seldinger technique. Guidewires are often used to facilitate intravascular access to gain access to target vessels for treatment such as stenting and aneurysm bypass grafting. During these procedures, the availability of the right guidewire for the task is essential.

### BRIEF SUMMARY

The present disclosure is directed to a guidewire with controllable stiffness. In one embodiment, the guidewire includes a proximal section, a distal section and a middle section between the proximal section and distal section. The distal section further includes a core wire, a shape memory coil, and a distal element electrically connecting a distal end of the core wire to a distal end of the shape memory coil. The shape memory coil has an expanded configuration having a first stiffness and a compressed configuration having a second stiffness.

Another embodiment of the present disclosure discloses a shape memory coil. The shape memory coil has a shape memory wire having a non-round cross section profile so that the shape memory coil has one or more stable curvatures. The shape memory coil has an expanded configuration having a first stiffness and a compressed configuration having a second stiffness.

The present disclosure also discloses a method of using a guidewire with controllable stiffness. The method includes providing a guidewire, which includes a proximal section, a distal section, and a middle section disposed between the proximal section and the distal section. The distal section includes a core wire, a shape memory coil, and a distal element electrically connecting a distal end of the core wire to a distal end of the shape memory coil. The shape memory coil has an expanded configuration having a first stiffness and a compressed configuration having a second stiffness. The method further includes applying an electric current to the shape memory coil for a duration to control a stiffness of the distal section.

### BRIEF DESCRIPTION OF THE DRAWINGS

FIG. 1 is a schematic diagram of a guidewire with controllable stiffness.

FIG. 2 is a schematic diagram of a distal section of a guidewire with controllable stiffness in one embodiment.

FIG. 3 is a schematic diagram of a distal section of a guidewire with controllable stiffness in another embodiment.

FIG. 4 is a schematic diagram of a distal section of a guidewire with controllable stiffness in one embodiment.

FIG. 5 is a schematic diagram of a distal section of a guidewire with controllable stiffness in another embodiment.

FIG. 6A is a schematic diagram of a distal section of a guidewire with controllable stiffness including more than one shape memory coils in serials in one embodiment.

FIG. 6B is a schematic diagram of a distal section of a guidewire with controllable stiffness including more than one shape memory coils in parallel in one embodiment.

FIG. 7A is a schematic diagram of a distal section of a guidewire with controllable stiffness including two shape memory coils in one embodiment.

FIG. 7B is a schematic diagram of a distal section of a guidewire with controllable stiffness including two shape memory coils in another embodiment.

FIG. 8 is a schematic diagram of a distal section of a guidewire with controllable stiffness when the core wire may retract in one embodiment.

FIG. 9 is a schematic diagram of a distal section of a guidewire with controllable stiffness when the stiffness and shape may be independently controlled in one embodiment.

FIG. 10A is a schematic diagram of a cross section view of a coil along the longitudinal axis of the coil when the coil is straight in one embodiment.

FIG. 10B is a schematic diagram of a cross section view of a coil along the longitudinal axis of the coil when the coil is in a preferred and stable curvature in one embodiment.

### DETAILED DESCRIPTION OF THE DRAWINGS

The invention will now be described in detail hereinafter with reference to the accompanied drawings, which form a part of the present invention, and which show, by way of illustration, specific examples of embodiments. Please note that the invention may, however, be embodied in a variety of different forms and, therefore, the covered or claimed subject matter is intended to be construed as not being limited to any of the embodiments to be set forth below. Please also note that the invention may be embodied as methods, devices, components, or systems. Accordingly, embodiments of the invention may, for example, take the form of hardware, software, firmware or any combination thereof.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter includes combinations of exemplary embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” or “at least one” as used herein, depending at least in part upon context, may be used to describe any feature, structure, or characteristic in a singular sense or may be used to describe combinations of features, structures or characteristics in a plural sense. Similarly, terms, such as “a”, “an”, or “the”, again, may be understood to convey a singular usage or to convey a plural usage, depending at least in part upon context. In addition, the term “based on” or “determined by” may be understood as not necessarily intended to convey an exclusive set of factors and may, instead, allow for existence of additional factors not necessarily expressly described, again, depending at least in part on context.

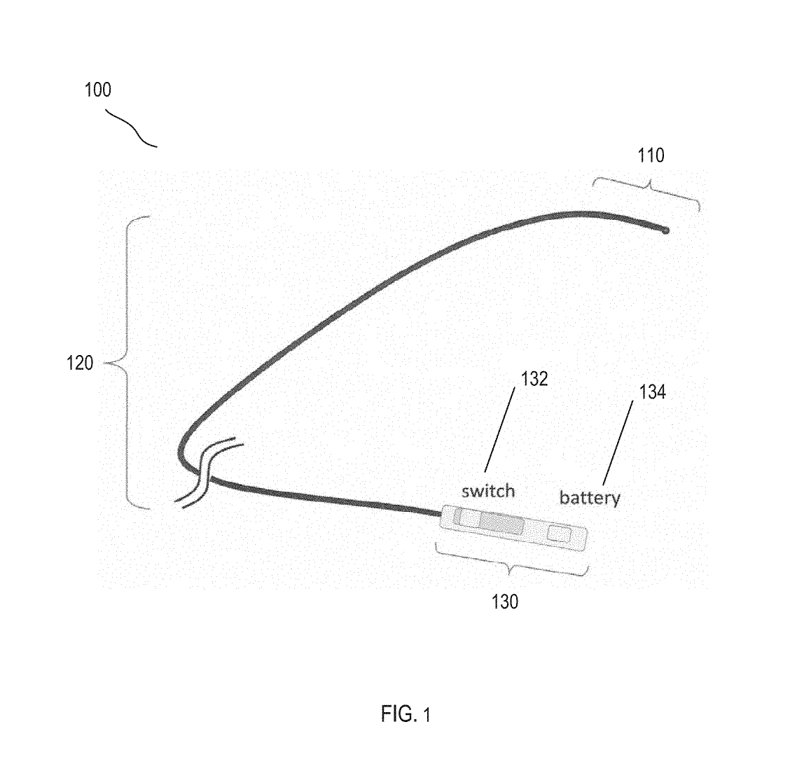

FIG. 1 shows a schematic diagram of a guidewire 100, which can be controlled to have different degrees of flexibility or stiffness during its application. In some embodiments, the guidewire may be controlled to have different degrees of curvature or length.

The guidewire 100 includes a proximal section 130, a distal section 110, and a middle section 120 between the proximal section 130 and the distal section 110. The proximal section 130 may electrically connect to the distal section 110.

The proximal section 130 may include a power supply component 134, for example but not limited to, a battery or a transformer. The power supply component 134 can provide electricity to the distal section 110. The electricity may be in a constant current or a pulsatile current.

The proximal section 130 may also include a power control device 132, for example but not limited to, a switch or a current regulating device. The power control device 132 may control a magnitude and a duration of the electricity passing from the power supply component 134 to the distal section 110.

In some embodiments, the power control device 132 may be remotely controlled by a user or an electronic device through either wireless or wired communication. For example but not limited to, the power control device 132 may be in communication wirelessly with a computer, and receive instructions or control signals from the computer to control the electricity from the power supply component 134 to the distal section 110.

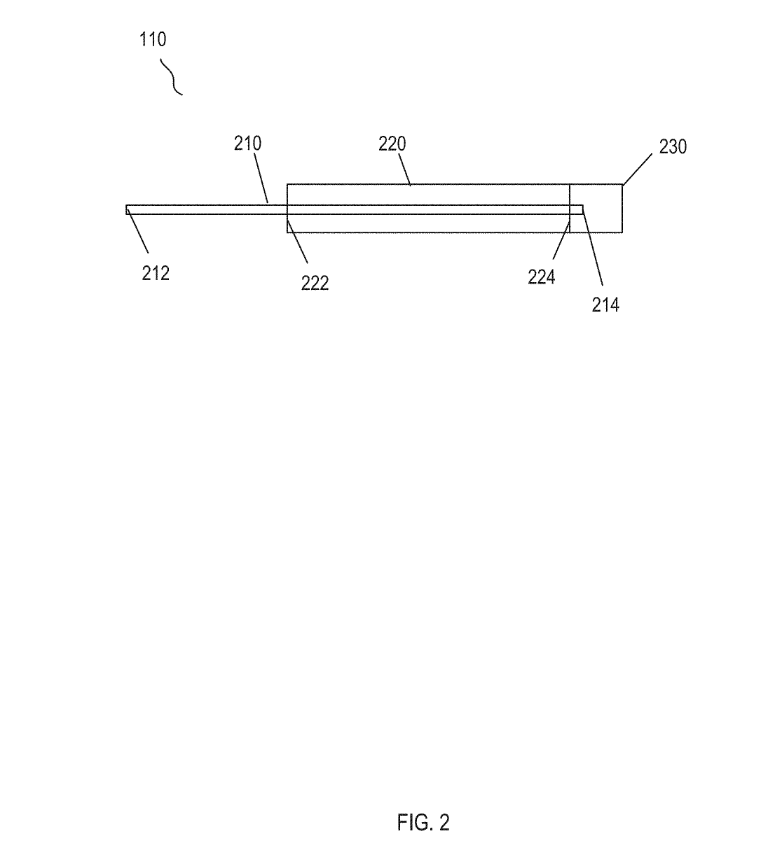

FIG. 2 shows a schematic diagram of a distal section 110 of a guidewire in one embodiment. The distal section 110 includes a core wire 210, a shape memory coil 220, and a distal element 230. The distal element 230 electrically connects a distal end 214 of the core wire 210 and a distal end 224 of the shape memory coil 220. A proximal end 222 of the shape memory coil 220 electrically connects to the proximal section of the guidewire. In some embodiment, the distal element 230 may be a solder joint.

The core wire 210 has a proximal end 212 and a distal end 214. The core wire 210 feeds through the shape memory coil 220. The distal end 214 of the core wire 210 is electrically connected to the distal element 230. The proximal end 212 of the core wire 210 is electrically connected to the power control component 132 and the power supply component 134.

The shape memory coil 220 has a proximal end 222 and a distal end 224. The proximal end 222 is electrically connected to the power control component 132 and the power supply component 134. The distal end 224 is electrically connected to the distal element 230.

The shape memory coil 220 is made of a shape memory wire, whose function is based on the shape memory phenomenon. The shape memory phenomenon occurs when a material forms a crystal structure and the crystal structure is capable of undergoing a change from one crystal form to another at a temperature determined by the material.

In one embodiment, the shape memory wire is made of a nickel titanium alloy. When both nickel and titanium atoms are present in the nickel titanium alloy in a predetermined ratio, the nickel titanium alloy forms a crystal structure above a predetermined temperature and is high strength and not easily deformed. The nickel titanium alloy forms another crystal structure below the predetermined temperature.

The predetermined temperature may be the transformation temperature. When the temperature cycle repeats, the strength or the shape of the nickel titanium alloy repeats as well. The temperature of the nickel titanium alloy can be controlled by the environmental temperature, heating, and/or cooling. Heating includes but not limited to electric heating and radiation heating. When an electric current passes through the nickel titanium alloy, a certain amount of heat is generated due to the resistance of the nickel titanium alloy. The heat generation can be controlled by controlling the magnitude and duration of the electrical current. Cooling includes but not limited to air/liquid cooling, passive cooling, and heat pipe cooling.

The transformation temperature of the nickel titanium alloy may be adjusted from over 100 Celsius degrees to below 0 Celsius degrees. In some embodiments, the transformation temperature of the shape memory wire is selected to be between 40 and 120 Celsius degrees. The heating to the shape memory wire is provided by controlling an electric current passing through the shape memory wire. Because the transformation temperature is above room temperature or the normal physiological temperature, the cooling to the shape memory wire may be provided by heat dissipation to its environment, for example, surrounding air, liquid, or blood inside vessels.

In some embodiments, to avoid thermal burning when a temperature of the shape memory coil is above the normal physiological temperature, the shape memory coil could be insulated by coating one or more layers of thermic insulation material. The thermic insulation material may include but not limited to polyurethane and aerogel. A proper thickness of a thermic insulation may be determined considering the transformation temperature and the specific application of the guidewire. With thinner thermic insulation, the cooling of the shape memory coil is larger so that the shape memory coil can cool down faster. With thicker thermic insulation, the cooling of the shape memory coil is decreased so that the shape memory coil may be heat up more quickly.

In some embodiments, the shape memory wire is made of FLEXINOL®, which is a trade name for shape memory wires made of nickel titanium alloy by Dynalloy Inc (1562 Reynolds Avenue, Irvine, Calif. 92614).

The shape memory coil 220 has an expanded configuration and a compressed configuration. When the shape memory coil 220 is in the expanded configuration, the shape memory coil has a first stiffness and a first length. When the shape memory coil 220 is in the compressed configuration, the shape memory coil has a second stiffness and a second length. In one embodiment, the first stiffness is lesser than the second stiffness. The first length is longer than the second length. In one embodiment, below the transformation temperature of the shape memory coil, the shape memory coil assumes the expanded configuration; and above the transformation temperature, the shape memory coil assumes the compressed configuration.

To avoid electric shock to users or to avoid electric leakage, the shape memory wire, the distal element 230 or the core wire 212 are electrically insulated (for example, enameled) from the surround environment.

In one embodiment, a first current passes through the shape memory coil 220 for a first duration so that the temperature of the shape memory coil 220 is below the transformation temperature and the shape memory coil assumes the expanded configuration. When a second current passes through the shape memory coil 220 for a second duration, the temperature of the shape memory coil 220 is above the transformation temperature and the shape memory coil assumes the compressed configuration. The first current may be lesser than the second current. In some embodiments, the first duration may be shorter than the second duration. In other embodiments, the first current may be zero, i.e., there is no current passing through the shape memory coil 220 so that the shape memory coil assumes the expanded configuration. The first or second current may be a direct current, an alternative current, or a variable current.

In one embodiment, the length of the shape memory coil 220 may be between 1 millimeter and 10 meters with a preferred length of about 10 millimeters. The overall outer diameter of the shape memory wire of the shape memory coil may be between 0.01 millimeters and 3.5 millimeters with a preferred overall outer diameter of about 0.5 millimeters.

In one embodiment, the shape memory coil 220 may bend downward when it is in the expanded configuration. The bending angle may be zero to 360 degrees, for example and not limited to, 5 degrees, 10 degrees, 30 degrees, 45 degrees, 90 degrees, and 180 degrees. When it is in the expanded configuration, the tip load of the shape memory coil 220, i.e., the weight needed to be applied to bend the distal section of the guidewire, may be between 0.5 grams and 15 grams. Preferably, in a floppy range, the tip load is equal to or smaller than 1 gram; in an intermediate range, the tip load is about 3 grams; and in a stiff range, the tip load is equal to or larger than 4.5 grams.

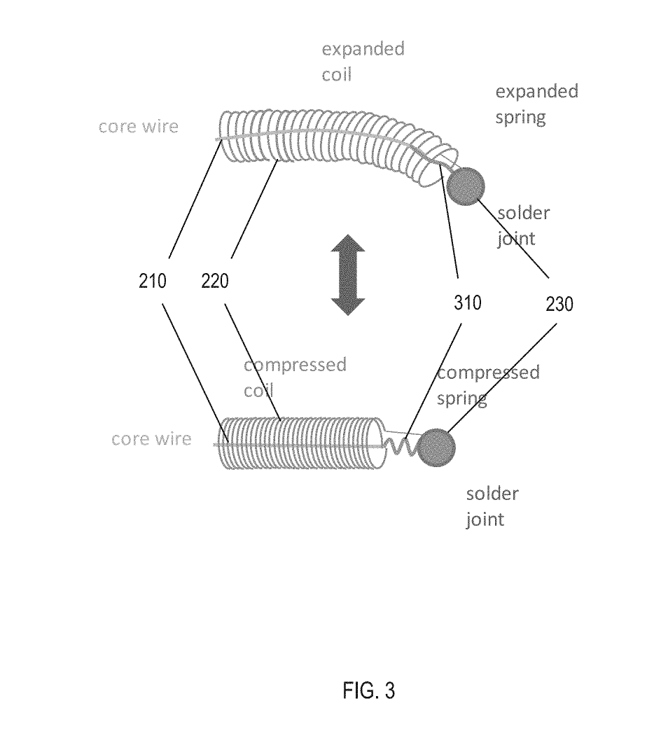

In one embodiment as in FIG. 3, a distal section of a guidewire includes a core wire 210, a shape memory coil 220, a spring 310, and a distal element 230. The distal element 230 electrically connects a distal end of the shape memory coil 220 to a distal end of the spring. A proximal end of the spring 310 is electrically connected to a distal end of the core wire 210. A proximal end of the core wire is electrically connected to a proximal section of the guidewire (not shown in FIG. 3).

The shape memory coil 220 has an expanded configuration and a compressed configuration. A user can control a magnitude or a duration of an electric current passing through the shape memory coil 220, so that the temperature of shape memory coil 220 may switch between below and above the transformation temperature, thus switching between the expanded configuration and the compressed configuration.

When the shape memory coil 220 is in the expanded configuration, the shape memory coil 220 is expanded and the spring 310 is expanded. In some embodiment, the shape memory coil 220 may bend with a curvature. The shape memory coil has a first stiffness. The distal section has a first length.

When the shape memory coil 220 is in the compressed configuration, the shape memory coil 220 is compressed and the spring 310 is compressed. The shape memory coil 220 may be straight. The shape memory coil has a second stiffness. The distal section has a second length. In some embodiments, the second stiffness is larger than the first stiffness. In other embodiment, the first length may be longer than the second length.

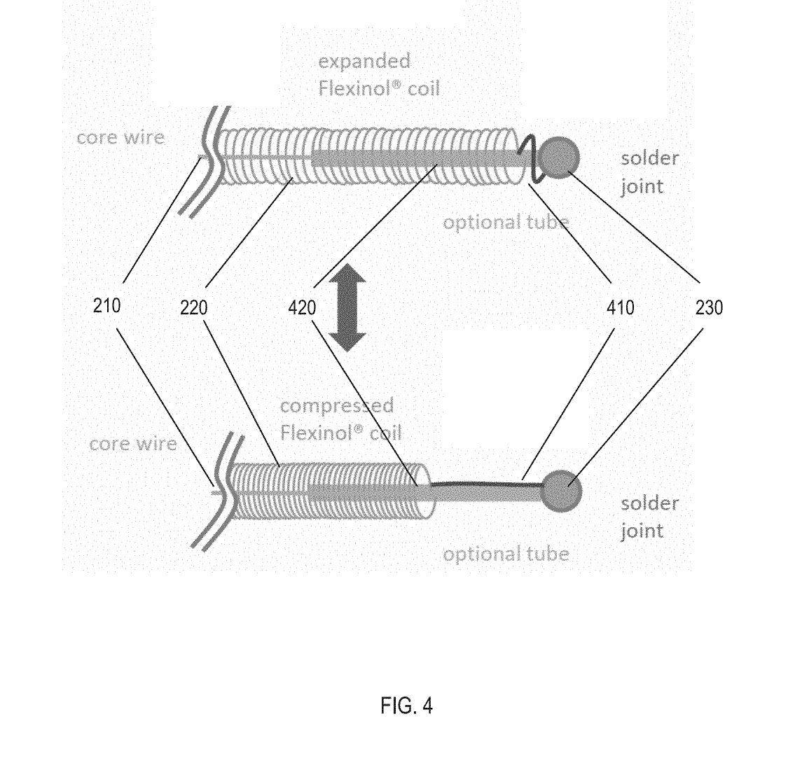

In one embodiment as in FIG. 4, a distal section of a guidewire includes a core wire 210, a shape memory coil 220, a spring 410, a tube 420, and a distal element 230. The distal element 230 electrically connects a distal end of the core wire 210 to a distal end of the spring 410. A proximal end of the spring 410 is electrically connected to a distal end of the shape memory coil 220. A proximal end of the shape memory coil 220 and a proximal end of the core wire are electrically connected to a proximal section of the guidewire (not shown in FIG. 4).

In some embodiment, the spring 410 may be an electrical conductive stretchable band. The electrical conductive stretchable band may be made from elastomer, for example and not limited to, rubber. The electrical conductive stretchable band electrically connects the distal element to the distal end of the shape memory coil 220. The electrical conductive stretchable band may have a relaxed configuration, a stretched configuration, or any configuration between the relaxed configuration and the stretched configuration.

The shape memory coil 220 has an expanded configuration and a compressed configuration. When a user may control a magnitude or a duration of an electric current passing through the shape memory coil 220, the shape memory coil 220 may switch between the expanded configuration and the compressed configuration.

The tube 420 encloses a distal portion of the core wire 210 and may be mechanically connected to the distal element 230. The tube 420 has a certain stiffness. An inner diameter of the tube 420 may be about the same as an outer diameter of the core wire 210, so that the tube may provide a certain strength and thus limit the flexibility of the distal section of the guidewire when the shape memory coil 220 is in the expanded configuration or the compressed configuration. For example but not limited to, when the shape memory coil 220 is in the compressed configuration, the tube 420 may be used to limit the flexibility of a tip of the guidewire so that the tip does not bend.

When the shape memory coil 220 is in the expanded configuration, the shape memory coil 220 is expanded and the spring 410 is compressed. In some embodiment, the shape memory coil 220 may bend with a curvature. The shape memory coil 220 has a first stiffness. The distal section has a first length. In some embodiment, the electrical conductive stretchable band may be in the relaxed configuration.

When the shape memory coil 220 is in the compressed configuration, the shape memory coil 220 is compressed and the spring 410 is expanded. The shape memory coil 220 may be straight and have a second stiffness. The distal section has a second length. In some embodiment, the second stiffness is larger than the first stiffness, and the first length and the second length are the same. In some embodiment, the electrical conductive stretchable band may be in the stretched configuration.

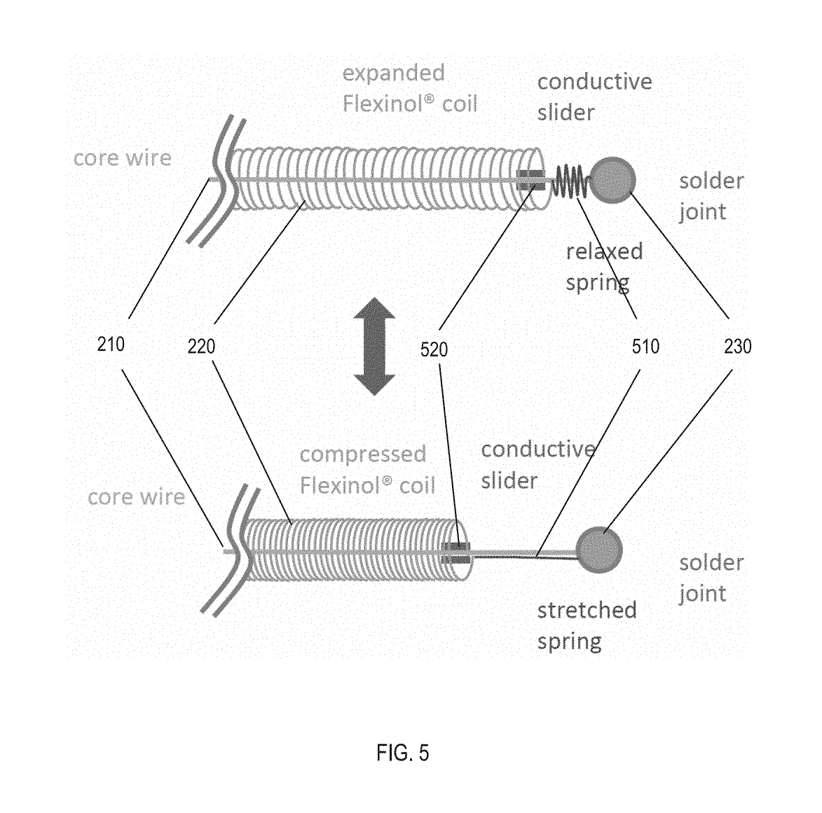

In one embodiment as in FIG. 5, a distal section of a guidewire includes a core wire 210, a shape memory coil 220, a spring 510, a slider 520, and a distal element 230. The distal element 230 electrically connects a distal end of the core wire 210 to a distal end of the spring 510. A proximal end of the spring 510 is electrically connected to a distal end of the shape memory coil 220. A proximal end of the shape memory coil 220 and a proximal end of the core wire 210 are electrically connected to a proximal section of the guidewire (not shown in FIG. 5).

The shape memory coil 220 has an expanded configuration and a compressed configuration. When a user controls a magnitude or a duration of an electric current passing through the shape memory coil 220, the shape memory coil 220 may switch between the expanded configuration and the compressed configuration.

The slider 520 is slidable and engagable along the core wire 210. The slider 520 is mechanically fixed to the distal end of the shape memory coil 220. The slider 520 provides mechanical strength to a tip of the distal section of the guidewire.

When the shape memory coil 220 is in the expanded configuration, the shape memory coil is expanded and the spring 510 is compressed. In some embodiment, the shape memory coil 220 may bend with a curvature. The shape memory coil 220 has a first stiffness. The distal section has a first length.

When the shape memory coil 220 is in the compressed configuration, the shape memory coil 220 is compressed and the spring 510 is expanded or stretched. The shape memory coil 220 may be straight. The shape memory coil has a second stiffness. The distal section has a second length. In some embodiment, the second stiffness is larger than the first stiffness, and the first length and the second length are the same.

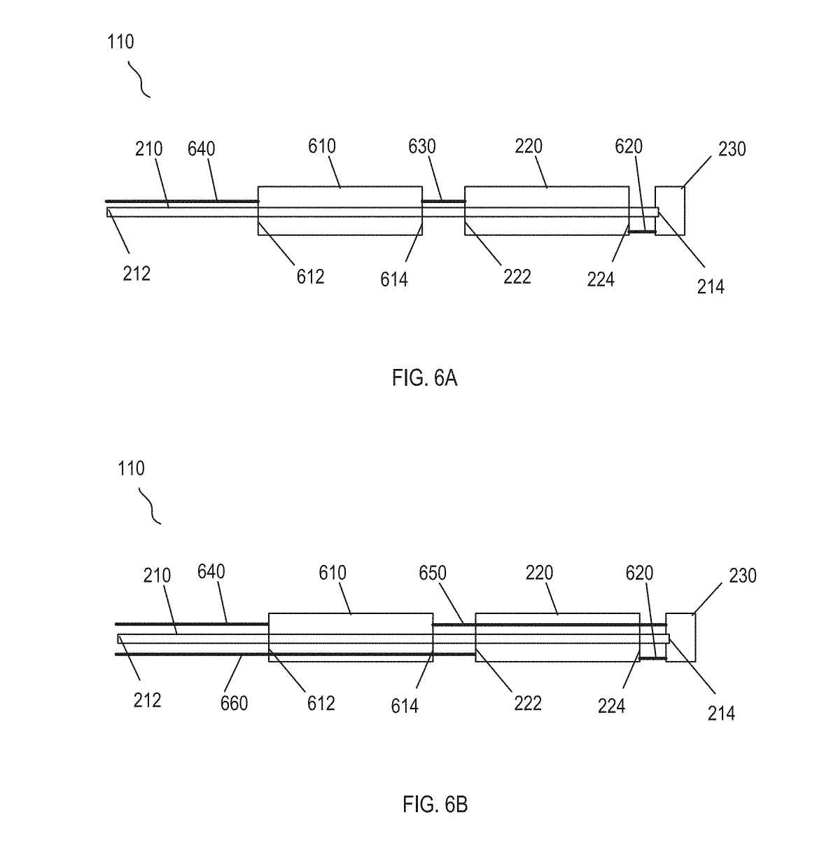

In some embodiments as in FIGS. 6A and 6B, the distal section 110 of the guidewire may include more than one shape memory coils. The more than one shape memory coils may be electrically connected in series as the two shape memory coils 220 and 610 in FIG. 6A, or may be electrically connected in parallel as the two shape memory coils 220 and 610 in FIG. 6B, so that the more than one shape memory coils may switch between a compressed configuration and a expanded configuration independently or together.

In one embodiment as in FIG. 6A, the distal section 110 of the guidewire includes two shape memory coils, a first shape memory coil 220 and a second shape memory coil 610. The distal section 110 also includes a core wire 210 and a distal element 230 electrically connecting to a distal end of the core wire 210. A first electrically conductive element 620 connects the distal element 230 to a distal end 224 of the first shape memory coil 220. A second electrically conductive element 630 connects a proximal end 222 of the first shape memory coil 220 to a distal end 614 of the second shape memory coil 610. A third electrically conductive element 640 connects a proximal end 612 of the second shape memory coil 610 to a proximal section of the guidewire (not shown in FIG. 6A).

The first shape memory coil 220 has an expanded configuration having a first stiffness and a compressed configuration having a second stiffness. The second shape memory coil 610 has an expanded configuration having a third stiffness and a compressed configuration having a fourth stiffness. A user may control an electric current passing through the first and second shape memory coils at the same time.

In one embodiment as in FIG. 6A, the current passing through the first shape memory coil 220 and the second shape memory coil 610 is the same, so that the first shape memory coil 220 and the second shape memory coil 610 are in the expanded configuration or the compressed configuration together.

In another embodiment as in FIG. 6A, the first shape memory coil 220 and the second shape memory coil 610 may have different transformation temperatures, so that when a first current passes the first and second shape memory coils for a first duration, the two shape memory coils assume the expanded configuration. When a second current passes through the two shape memory coils, one of the two shape memory coils assumes the compressed configuration while the other is in the expanded configuration. When a third current passes the first and second shape memory coils for a third duration, the two shape memory coils assume the compressed configuration.

In another embodiment as in FIG. 6A, when a first current passes the first and second shape memory coils for a first duration, the two shape memory coils may reach different temperatures. This may be due to many factors, for example and not limited to, the different cooling of the two shape memory coils and the different electric resistance of the two shape memory coils. Therefore, one of the two shape memory coils assumes the compressed configuration while the other is in the expanded configuration. When a second current passes the first and second shape memory coils for a second duration, the two shape memory coils assume the compressed configuration.

In one embodiment as in FIG. 6B, a distal section 110 of a guidewire includes two shape memory coils, a first shape memory coil 220 and a second shape memory coil 610. The distal section 110 also includes a core wire 210 and a distal element 230 electrically connecting to a distal end of the core wire. A first electrically conductive element 620 connects the distal element 230 to a distal end 224 of the first shape memory coil 220. A fourth electrically conductive element 650 connects a distal end 614 of the second shape memory coil 610 to the distal element 230. A third electrically conductive element 640 connects a proximal end 612 of the second shape memory coil 610 to a proximal section of the guidewire (not shown in FIG. 6B). A fifth electrically conductive element 660 connects a proximal end 222 of the first shape memory coil 220 to the proximal section of the guidewire (not shown in FIG. 6B).

The first shape memory coil 220 has an expanded configuration having a first stiffness and a compressed configuration having a second stiffness. The second shape memory coil 610 has an expanded configuration having a third stiffness and a compressed configuration having a fourth stiffness. A user can control an electric current passing through the first and second shape memory coils individually, so that the first and second shape memory coils may switch between the expanded configuration and the compressed configuration independently.

In one embodiment as in FIG. 6B, the current passing through the first shape memory coil 220 and the second shape memory coil 610 is different, so that the first shape memory coil 220 and the second shape memory coil 610 are in the expanded configuration or the compressed configuration independently from each other.

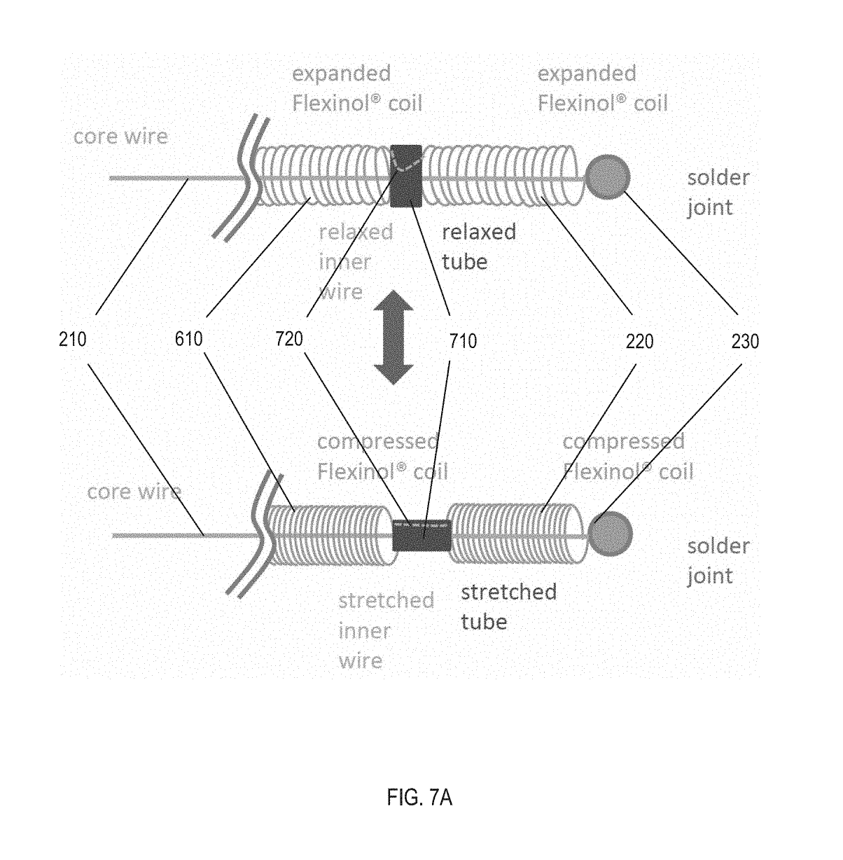

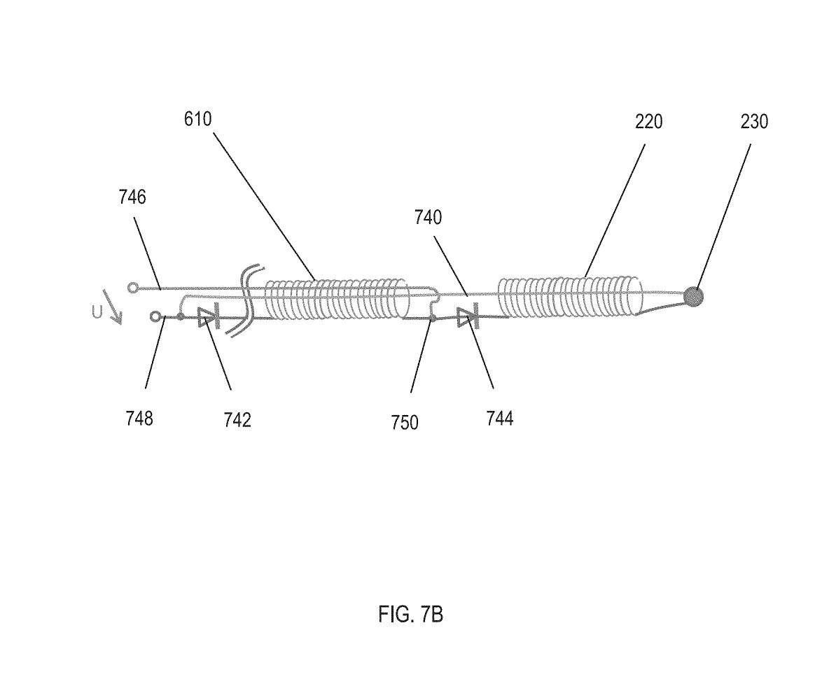

In one embodiment as in FIG. 7A, a distal section of a guidewire includes a core wire 210, a first shape memory coil 220, a second shape memory coil 610, an inter-coil connector, and a distal element 230. The inter-coil connector may include a tube 710 and an inner wire 720. The distal element 230 electrically connects a distal end of the core wire 210 to a distal end of the first shape memory coil 220. The tube 710 mechanically connects a proximal end of the first shape memory coil 220 to a distal end of the second shape memory coil 610, so that the tube provides mechanical strength to support and connect the two shape memory coils. In one embodiment, the inner wire 720 electrically connects the proximal end of the first shape memory coil 220 to the distal end of the second shape memory coil 610. A proximal end of the second shape memory coil 610 and a proximal end of the core wire 210 are electrically connected to a proximal section of the guidewire (not shown in FIG. 7A). In some embodiment, the tube 710 may be a spring.

When a first current passes through the first and second shape memory coils for a first duration, the two shape memory coils are in their expanded configurations having a first stiffness and a third stiffness, respectively. The distal section of the guidewire has a first length. The inner wire 720 is in a relaxed configuration and the tube 710 is in a relaxed configuration.

When a second current passes through the first and second shape memory coils for a second duration, the two shape memory coils are in their compressed configurations having a second stiffness and a fourth stiffness, respectively. The distal section of the guidewire has a second length. The inner wire 720 is in a stretched configuration and the tube 710 is in a stretched configuration. In one embodiment, the second stiffness is larger than the first stiffness and the fourth stiffness is larger than the third stiffness. In some embodiment, the first length is the same as the second length. The second current is larger than the first current or the second duration is longer than the first duration.

In another embodiment as in FIG. 7B, a distal section of a guidewire includes a core wire 740, a first shape memory coil 220, a second shape memory coil 610, a distal element 230, a first wire 746, and a second wire 748. The first and second shape memory coils are electrically connected by a first diode 744. The distal element 230 electrically connects a distal end of the core wire 740 to a distal end of the first shape memory coil 220. A distal end of the first wire 746 connects to a distal end 750 of the second shape memory coil 610. A distal end of the second wire 748 electrically connects to the core wire 740, and electrically connects to the second shape memory 610 via a second diode 742. A proximal end of the first wire 746 and a proximal end of the second wire 748 electrically connect to a proximal section of the guidewire (not shown in FIG. 7B).

The polarity of the first and second diodes are chosen and disposed in the distal section of the guidewire, so that when positive or negative voltages are applied to the proximal ends of the first and second wires, electrical current passes through either the first shape memory coil 220 or the second shape memory coil 610. Thus, the expanded/compressed configurations of the first/second shape memory coils may be controlled independently.

In the embodiment as in FIG. 7B, the first diode 744 is disposed so that an electric current may only flow from a proximal end of the first shape memory coil 220 to a distal end of the first shape memory coil 220. The second diode 742 is disposed so that an electric current may only flow from the proximal end of the second shape memory coil 610 to the distal end of the second shape memory coil 610. Therefore, when a positive voltage is applied between the proximal end of the first wire 746 and the proximal end of the second wire 748, an electric current passes through the first shape memory coil but not through the second shape memory coil, so that the first shape memory coil may be in its compressed configuration and the second shape memory coil may be in its expanded configuration.

When a negative voltage is applied between the proximal end of the first wire 746 and the proximal end of the second wire 748, an electric current passes through the second shape memory coil but not through the first shape memory coil, so that the second shape memory coil may be in its compressed configuration and the first shape memory coil may be in its expanded configuration.

When no voltage is applied between the proximal end of the first wire 746 and the proximal end of the second wire 748, there is no electric current passing through the first and second shape memory coils, so that both shape memory coils may be in the expanded configurations.

In some embodiment as in FIG. 2, a core wire 210 of a guidewire may be pulled towards a proximal direction relative to a distal section 110 of the guidewire, so that the distal section 110 of the guidewire becomes shorter or transitions into a stiffer state. In other embodiments, the core wire 210 of the guidewire may be relaxed or pushed towards a distal direction relative to the distal section 110 of the guidewire, so that the shape memory coil 220 may be expended or the distal section 110 of the guidewire assumes a bent and curved shape.

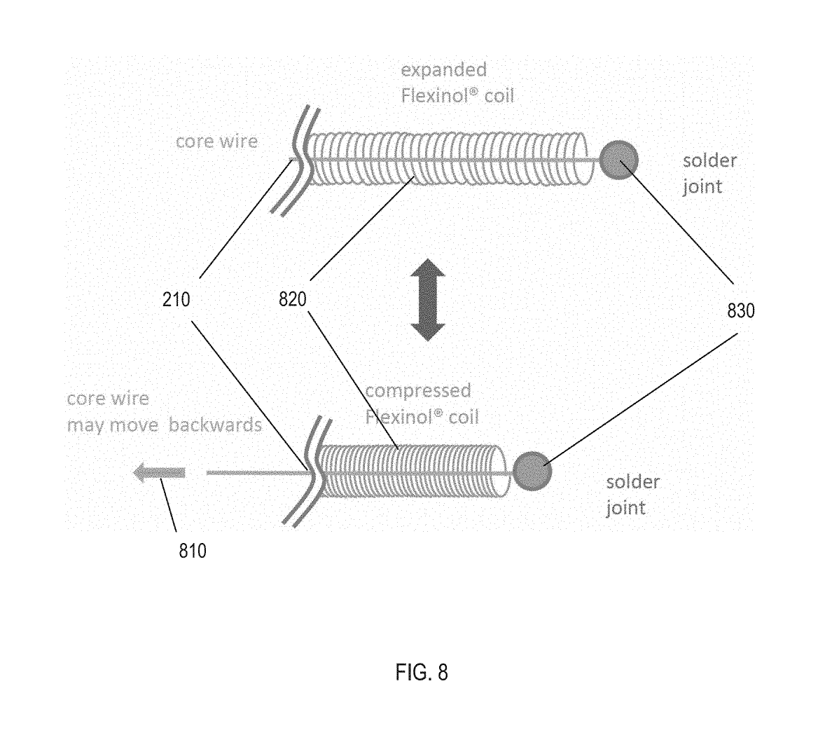

In one embodiment as in FIG. 8, a core wire 210 of a guidewire is retracted towards a proximal direction 810 so that a distal section of the guidewire becomes shorter and stiffer. The distal section of the guidewire may include a coil 820 and a distal element 830.

In some embodiment, the coil 820 may be a regular mechanical coil. When the core wire 210 is relaxed or pushed toward a distal direction, the regular mechanical coil 820 is in a relaxed state, which may be straight or bend with a curvature. When the core wire 210 is retraced towards the proximal direction 810, the regular mechanical coil 820 is compressed by a distal element 830 of the distal section.

In some embodiment, the coil 820 may be a shape memory coil. The distal element 830 may electrically connect a distal end of the core wire 210 to a distal end of the shape memory coil 820. When a first current passes the shape memory coil for a first duration, the shape memory coil assumes an expanded configuration with a first stiffness. When a second current passes the shape memory coil for a second duration, the shape memory coil assumes a compressed configuration with a second stiffness, where the second stiffness is larger than the first stiffness.

In some embodiment, the stiffness and compression/expansion state of the shape memory coil 820 controlled by passage of current may work together with the retraction of the core wire 210 in a particular sequence or work independently.

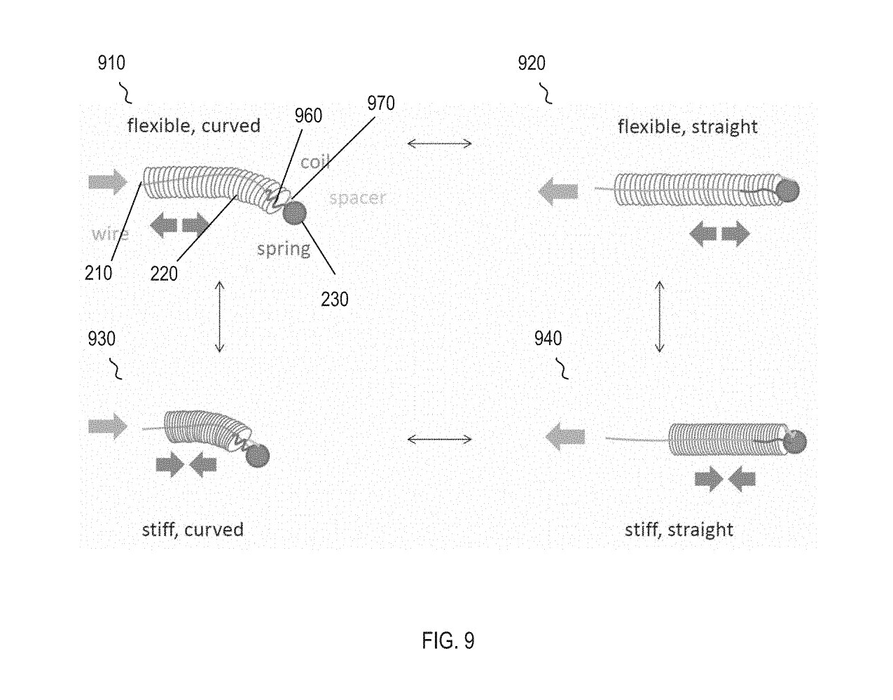

In one embodiment in FIG. 9, a stiffness and a shape of a distal section of a guidewire may be independently controlled. The distal section of the guidewire includes a core wire 210, a shape memory coil 220, a spring 960, a spacer 970, and a distal element 230. The distal element 230 electrically connects a distal end of the space 970 and a distal end of the spring 960. A proximal end of the space 970 is connected to a distal end of the shape memory coil 220. A proximal end of the spring 960 is electrically connected to a distal end of the core wire 210. A proximal end of the shape memory coil 220 and a proximal end of the core wire are electrically connected to a proximal section of the guidewire (not shown in FIG. 9). The core wire 210 may be retracted towards a proximal direction so that the shape of the distal section becomes straight. The independent passage of an electric current through the shape memory coil 220 may provide an independent control of the stiffness and shape of the distal section of the guidewire. The spring 960 may have a relaxed configuration, a stretched configuration, or any configuration between the relaxed configuration and the stretched configuration.

When the core wire 210 is relaxed or pushed towards a distal direction and the shape memory coil 220 is in an expanded configuration, the distal section of the guidewire may be curved and flexible as in 910. When the core wire 210 is retracted towards the proximal direction and the shape memory coil 220 is in the expanded configuration, the distal section of the guidewire may be straight and flexible as in 920. When the core wire 210 is relaxed or pushed towards a distal direction and the shape memory coil 220 is in a compressed configuration, the distal section of the guidewire may be curved and stiff as in 930. When the core wire is retracted towards the proximal direction and the shape memory coil 220 is in the compressed configuration, the distal section of the guidewire may be straight and stiff as in 940.

In some embodiments, a wire with a non-round cross section profile may be used to make a coil. The non-round cross section profile may include but not limited to triangle, square, rectangle, pentagon, hexagon, and octagon. When the coil bends, it has one or more preferred and stable angles, providing stabilized specific bending curvatures for the coil. A shape memory coil may be made from a shape memory wire having a non-round cross section profile so that the shape memory coil has one or more stable curvatures. The shape memory coil with one or more stable curvatures may be used in guidewires in the previous embodiments.

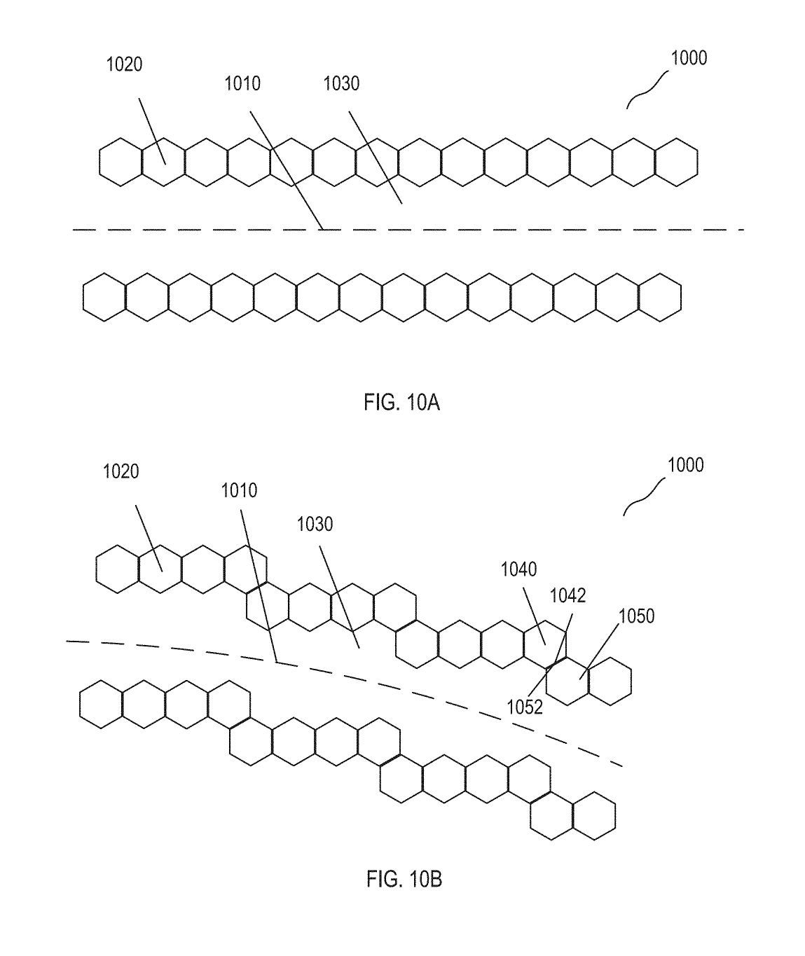

FIG. 10A depicts a cross section view of a coil 1000 along its longitudinal axis 1010 in one embodiment when the coil is straight. The coil 1000 encloses an inner space 1030. The coil is made from a wire having a hexagonal cross section profile 1020.

FIG. 10B depicts a cross section view of a coil 1000 along its longitudinal axis 1010 in one embodiment when the coil is in one of preferred angles. For a first cross section 1040 of the wire and a second cross section 1050 of the wire, a side 1042 of the first section 1040 is preferred to contact with a side 1052 of the second section 1050, resulting a preferred and stable curvature of the coil 1000. In some embodiment, the non-round cross section profile of the wire may be designed in order to achieve a particular preferred curvature.

While the particular invention has been described with reference to illustrative embodiments, this description is not meant to be limiting. Various modifications of the illustrative embodiments and additional embodiments of the invention will be apparent to one of ordinary skill in the art from this description. Those skilled in the art will readily recognize that these and various other modifications can be made to the exemplary embodiments, illustrated and described herein, without departing from the spirit and scope of the present invention. It is therefore contemplated that the appended claims will cover any such modifications and alternate embodiments. Certain proportions within the illustrations may be exaggerated, while other proportions may be minimized. Accordingly, the disclosure and the figures are to be regarded as illustrative rather than restrictive.

1. A guidewire, comprising:
a proximal section;
a middle section;
a distal section comprising:
a core wire,
a shape memory coil, and
a distal element electrically connecting a distal end of the core wire to a distal end of the shape memory coil, wherein:
the shape memory coil comprises an expanded configuration having a first stiffness and a compressed configuration having a second stiffness; and
wherein the middle section is disposed between the proximal section and the distal section.

2. The guidewire of claim 1, wherein:
the first stiffness is lesser than the second stiffness.

3. The guidewire of claim 1, wherein:
the shape memory coil assumes the expanded configuration upon passage of a first electric current for a first duration; and
the shape memory coil assumes the compressed configuration upon passage of a second electric current for a second duration, wherein the first electric current is lesser than the second electric current or the first duration is shorter than the second duration.

4. The guidewire of claim 1, wherein:
the shape memory coil comprises a shape memory wire enameled by electrical insulation material; and
the shape memory coil is coated with a layer of thermic insulation material.

5. The guidewire of claim 1, wherein:
the distal section further comprises a spring electrically connecting the distal element to the distal end of the core wire;
when the shape memory coil is in the expanded configuration, the spring is in an expanded state and the distal section has a first length; and
when the shape memory coil is in the compressed configuration, the spring is in a compressed state and the distal section has a second length, the first length being longer than the second length.

6. The guidewire of claim 1, wherein:
the distal section further comprises a tube surrounding a portion of the core wire and an electrical conductive stretchable band electrically connecting the distal element to the distal end of the shape memory coil;
a distal end of the tube mechanically connects to the distal element;
when the shape memory coil is in the expanded configuration, the electrical conductive stretchable band is in a relaxed state and the distal section has a first length; and
when the shape memory coil is in the compressed configuration, the electrical conductive stretchable band is in a stretched state and the distal section has a second length, the first length being equal to the second length.

7. The guidewire of claim 1, wherein:
the distal section further comprises a slider slidably engagable along the core wire and a spring electrically connecting the distal element to the shape memory coil;
the slider is mechanically fixed to the distal end of the shape memory coil;
when the shape memory coil is in the expanded configuration, the spring is in a compressed state and the distal section has a first length; and
when the shape memory coil is in the compressed configuration, the spring is in an expanded state and the distal section has a second length, the first length being equal to the second length.

8. The guidewire of claim 1, wherein:
when the shape memory coil is in the expanded configuration, the shape memory coil is curved; and
when the shape memory coil is in the compressed configuration, the shape memory coil is straight.

9. The guidewire of claim 1, wherein:
the shape memory coil comprises a nickel titanium alloy;
the shape memory coil comprises a length between 1 millimeter and 10 meters; and
the shape memory coil comprises a collective outer diameter between 0.01 millimeter and 3.5 millimeter.

10. The guidewire of claim 1, further comprising:
a power device comprising a power control device and a power supply component, wherein:
the power device electrically connects to a proximal end of the core wire and a proximal end of the shape memory coil,
the power supply component provides electricity to the shape memory coil, and
the power control device controls a magnitude and a duration of the electricity to the shape memory coil.

11. The guidewire of claim 1, wherein the distal section further comprises:
a second shape memory coil;
a connector mechanically connecting the second shape memory coil to the shape memory coil; and
wherein:
the second shape memory coil comprises an expanded configuration and a compressed configuration,
when the second shape memory coil is in the expanded configuration, the second shape memory coil has a third stiffness,
when the second shape memory coil is in the compressed configuration, the second shape memory coil has a fourth stiffness, and
the third stiffness is lesser than the fourth stiffness.

12. The guidewire of claim 11, wherein:
the second shape memory coil assumes the expanded configuration upon passage of a third electric current for a third duration; and
the second shape memory coil assumes the compressed configuration upon passage of a fourth electric current for a fourth duration.

13. The guidewire of claim 11, wherein:
the second shape memory coil and the shape memory coil are electrically in parallel or in serial to each other.

14. The guidewire of claim 11, wherein:
the connector comprises an inner wire and an elastic device, wherein:
the inner wire electrically connects the second shape memory coil to the shape memory coil;
the elastic device mechanically connects the second shape memory coil to the shape memory coil;
when the second shape memory coil and the shape memory coil are in the expanded configuration, the inner wire and the elastic device are in a relaxed state and the distal section has a first length; and
when the second shape memory coil and the shape memory coil are in the compressed configuration, the inner wire and the elastic device are in a stretched state and the distal section has a second length, the first length being equal to the second length.

15. The guidewire of claim 11, wherein:
a first diode electrically connects to the shape memory coil and a second diode electrically connects to the second shape memory coil, so that:
when a first current passes through the shape memory coil, the first current does not pass through the second shape memory coil, and
when a second current passes through the second shape memory coil, the second current does not pass through the shape memory coil.

16. The guidewire of claim 1, wherein:
the core wire is movable relative to the shape memory coil between a first position and a second position;
when the core wire is in the first position, the shape memory coil is in the expanded configuration and curved, has a first stiffness, and has a first length; and
when the core wire is in the second position, the shape memory coil is in the compressed configuration and straight, has a second stiffness, and has a second length.

17. The guidewire of claim 1, wherein:
the shape memory coil comprises a shape memory wire having a hexagonal cross section profile.

18. The guidewire of claim 1, wherein the distal section further comprises:
a second coil connecting to the shape memory coil, and the second coil being a non-shape memory coil.

19. A shape memory coil, comprising:
a shape memory wire having a non-round cross section profile so that the shape memory coil has at least one stable curvature;
an expanded configuration having a first stiffness; and
a compressed configuration having a second stiffness, wherein the first stiffness is lesser than the second stiffness, and the non-round cross section profile of the shape memory wire is selected from the group consisting of triangle, square, rectangle, pentagon, hexagon, and octagon.

20. A method of using a guidewire, comprising:
providing a guidewire, the guidewire comprising:
a proximal section,
a distal section comprising:
a core wire,
a shape memory coil,
a distal element electrically connecting a distal end of the core wire to a distal end of the shape memory coil, wherein the shape memory coil comprises an expanded configuration having a first stiffness and a compressed configuration having a second stiffness, and
a middle section disposed between the proximal section and the distal section;
applying a first electric current to the shape memory coil for a first duration so that the shape memory coil assumes the expanded configuration; and
applying a second electric current to the shape memory coil for a second duration so that the shape memory coil assumes the compressed configuration.
